(19) United States
(12) Patent Application Publication
(10) Pub. No.: US 2006/0292641 A1
(43) Pub. Date: Dec. 28, 2006

(54) Immunoassay method

(76) Inventors: Hisao Nakanishi, Tokyo (JP); Takehiko Kitamori, Tokyo (JP)

Correspondence Address: SMITH, GAMBRELL & RUSSELL, SUITE 3100, PROMENADE II, 1230 PEACHTREE STREET, N.E., ATLANTA, GA, 30307-3592, US

(21) Appl. No.: 11/475,544

(22) Filed: Jun. 27, 2006

(30) Foreign Application Priority Data
Jun. 28, 2005 (JP) 2005-188222

(51) Int. Cl. G01N 33/53

(52) U.S. Cl. 435/7.5; 435/7.93

(57) ABSTRACT

The present invention provides an immunoassay method easier in operation and smaller in fluctuation of reaction. The immunoassay method according to the present invention is an immunoassay method, which employs a sandwich method using a microchip having an antibody immobilized in the micro-flow channel thereof, comprising feeding a fluid mixed with a labeled antibody and a specimen into the micro-flow channel.

This application is based on Japanese Patent application No.2005-188222, the content of which is incorporated herein by reference.

### BACKGROUND OF INVENTION

1. Technical Field

The present invention relates to an immunoassay method, which employs a sandwich method using a microchip having a micro-flow channel.

2. Related Art

Techniques for accelerating unit operations such as mixing, reaction, synthesis, extraction, separation, and analysis and for handling a smaller sample and for handling in microspace are attracting attention in the fields of organic chemistry and biochemistry, and thus, microchips have been under intensive investigation for establishment of such techniques.

The microchips are generally made of two glass substrates, one having a fine flow channel and the other having a hole for introducing a sample and a hole for discharging a sample, that are joined to each other.

When an immunoassay by sandwich method is performed in the microchip, an antibody capable of trapping a particular protein (hereinafter, referred to as marker) contained in a specimen is immobilized in a certain region of the flow channel. Then, the region where the antibody is not immobilized is coated with a protein having no influence on the assay reaction such as bovine serum albumin. The specimen is then fed therein so as to bring the specimen in contact with the antibody, and a buffer solution containing a surfactant is then fed to wash the internal flow channel. Then, an antibody specifically binding to the marker labeled with an enzyme, fluorescence dye, biotin, or the like is fed, and the flow channel is washed similarly. For example when an enzyme label is used, a liquid containing its substrate is fed to the channel, and color development of the substrate in the enzyme reaction is measured (Japanese Laid-open patent publication No. 2001-4628).

### SUMMARY OF THE INVENTION

In the immunoassay by the sandwich method described above, there are many kinds of sample liquids to be fed into the flow channel and thus, the operation is rather complicated. And such a procedure of feeding different sample liquids in different steps increases the possibility that foaming and impurities are mixed in the flow channel during switching of the steps, consequently, leading to fluctuation of the assay reaction.

An object of the present invention is to provide an immunoassay method easier in operation and smaller in the fluctuation of reaction in order to solve the problems above.

According to the present invention, there is provided

an immunoassay method, which employs a sandwich method using a microchip having an antibody immobilized in the micro-flow channel thereof, including feeding a fluid mixed with a labeled antibody and a specimen into the micro-flow channel; and

the immunoassay method described in (1) wherein the label is an enzyme, a fluorescent dye, or biotin.

According to the immunoassay method of the present invention, because a specimen and a labeled antibody are mixed before they are applied into the microchip, the steps of feeding a specimen, a washing solution and a labeled antibody, which have been performed separately, may be integrated into a single step. For that reason, it is possible to shorten the period needed for reaction and eliminate the troubles described above during reagent exchange.

Accordingly, the present invention provides an immunoassay method easier in operation and smaller in the fluctuation of reaction.

### BRIEF DESCRIPTION OF THE DRAWINGS

The above and other objects, advantages and features of the present invention will be more apparent from the following description taken in conjunction with the accompanying drawings, in which:

FIG. 1 is a schematic view illustrating an example of the microchip used in the present embodiment.

FIG. 2 is a schematic cross-sectional view of a dam in the micro-flow channel of the microchip in FIG. 1.

### DETAILED DESCRIPTION

The invention will be now described herein with reference to illustrative embodiments. Those skilled in the art will recognize that many alternative embodiments can be accomplished using the teachings of the present invention and that the invention is not limited to the embodiments illustrated for explanatory purposed.

Hereinafter, an embodiment of the present invention will be described.

The immunoassay method in the present embodiment is an immunoassay method, which employs a sandwich method using a microchip having an antibody immobilized in the micro-flow channel thereof, including feeding a fluid mixed with a labeled antibody and a specimen into the micro-flow channel.

In the present embodiment, a fluid containing a labeled antibody and a specimen is fed to the micro-flow channel, and the reactions between the immobilized antibody and a marker in the specimen and between the marker bound to an immobilized antibody and a labeled antibody proceed in the same step. Thus, it is possible after the reaction to quantify the labeled antibody which is reacted with the marker. The marker is previously bound to the immobilized antibody. It is possible to quantify the marker contained in the specimen from the quantification results, and thus, to determine immunologically the content of the marker in the fluid fed to the micro-flow channel.

In conventional immunoassays, a fluid containing a specimen is fed to a micro-flow channel to allow the marker in the specimen to react with an immobilized antibody therein and a fluid containing a labeled antibody is then fed to the same micro-flow channel, to allow the labeled antibody to react with the marker bound to the immobilized antibody during quantitative analysis. In such a case, the operation is more complicated, because it is necessary to feed the specimen, washing solution, and labeled antibody in separate steps and to feed a greater number of samples into the same flow channel. In addition, there is a high possibility that foaming and impurities are mixed in the flow channel between these steps, consequently leading to fluctuation of the reaction therein.

In contrast in the present embodiment, it is possible to integrate the steps of feeding the specimen, washing solution, and labeled antibody into a single step, because the labeled antibody and the specimen are fed to the micro-flow channel after they are previously mixed.

In addition in the present embodiment, intermolecular distances among the immobilized antibody, the marker in specimen, and the labeled antibody in the micro-flow channel seem to be significantly smaller than those in conventional assay methods. Thus, it seems to be possible to advance the reactions between the immobilized antibody and the marker and between the marker bound to the immobilized antibody and the labeled antibody more efficiently.

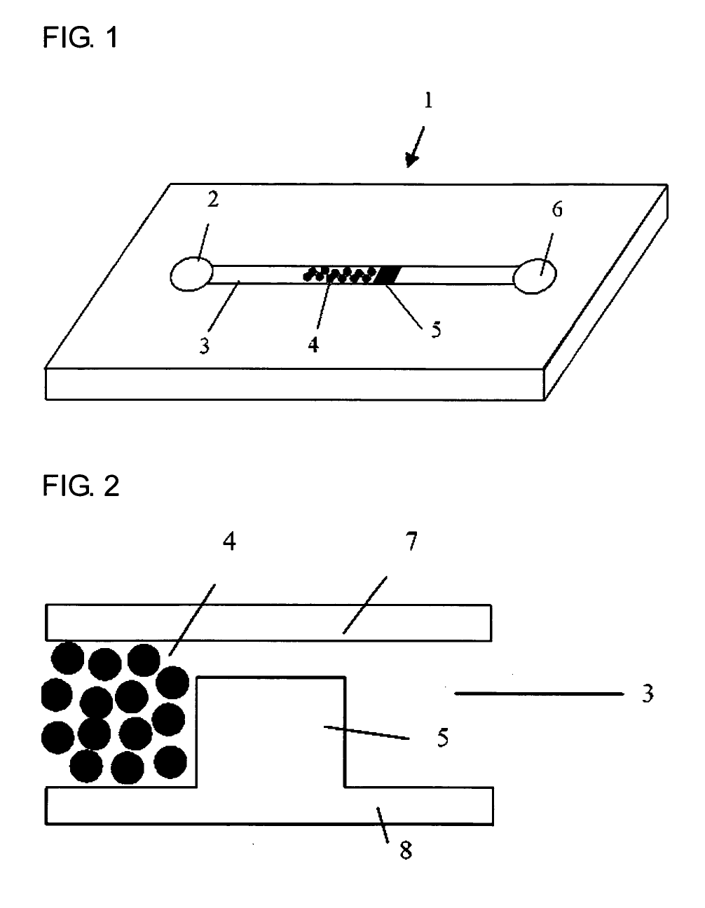

The immunoassay method in the present embodiment is achieved, for example, by using the microchip shown in FIG. 1 or 2.

The microchip 1 has a fluid inlet 2 for introducing a fluid, a micro-flow channel 3 in which the introduced fluid flows, and an outlet 6 for discharging the fluid flown through the micro-flow channel 3. It also has an antibody-immobilized reaction unit 4 placed midway in the micro-flow channel 3 and a dam 5 for controlling the flow of the fluid or stacking antibody-coated microbeads, installed downstream side of the reaction unit 4.

As shown in FIG. 2, the dam 5 is a wall-shaped material extending from the bottom 8 to an upper surface 7 in the micro-flow channel 3, and there is an opening allowing overflow of the fluid that is formed between the dam 5 and the upper surface 7 of the micro-flow channel 3. The dam makes the fluid remain in the reaction unit 4 to some extent, raising the efficiency of the reactions between antibody and marker and between marker and labeled antibody.

An antibody specifically binding to the marker contained in the specimen is immobilized in the micro-flow channel 3, forming an immobilized antibody. FIG. 2 shows a method of immobilizing an antibody on the surface of microbeads having a diameter of 10 to 100 μm and then introducing the microbeads into the micro-flow channel 3 having the dam structure. Although not shown in the figure, the antibody may be immobilized directly on the surface of the micro-flow channel. Both methods are applicable to the present invention. Accordingly, the distance between an antibody and a marker can be reduced in comparison to the conventional assay method, in which the distance between an antibody and a marker (antigen) is larger since the antibody is immobilized on the surface of the container.

The microbeads having the antibody thus immobilized are coated with an aqueous solution containing a protein less reactive with the immobilized antibody such as bovine serum albumin at the region on the bead surface where the antibody is not immobilized. The microbeads thus obtained are introduced to the reaction unit 4 of the micro-flow channel 3 to fill into the reaction unit 4 to a desirable amount. By such a coating treatment on the microbeads before filling, it is possible, when a fluid containing the labeled antibody is fed, to prevent nonspecific adsorption of the labeled antibody on the microbeads surface, i.e., immobilization of a labeled antibody on the exposed region other than the antibody-immobilized region, and to quantify accurately the labeled antibody bound to the marker that is previously bound to the immobilized antibody.

Separately, a solution containing a specimen and a solution containing a labeled antibody are taken out respectively in desirable amounts and are mixed and stirred in a container such as microtube in advance. The mixed solution thus obtained is fed to the micro-flow channel 3 and introduced to the reaction unit 4 to which the antibody-immobilized microbeads is previously introduced. If necessary, a washing solution containing a surfactant and the like is then introduced to the micro-flow channel 3 so as to wash the internal reaction unit 4. The micro-flow channel 3 preferably has a width of approximately 0.01 to 1 mm and a depth of 0.01 to 1 mm, in view of bringing the immobilized antibody, specimen, and labeled antibody sufficiently close to each other in the fluid, i.e., for reducing the intermolecular distance among them.

Examples of the labels in the labeled specimen include enzymes, fluorescence dyes, and biotin. When the label is an enzyme, after a mixed solution containing the specimen and labeled antibody is fed to the reaction unit 4, a solution containing a substrate is introduced thereto additionally to react with the labeled enzyme. The concentration of the marker contained in the specimen is determined by measuring the color development of the substrate colored by the reaction with the labeled enzyme by using a high-sensitivity colorimetric detector such as thermal lens microscope (hereinafter, referred to as TLM). Alternatively when the label is biotin, a solution containing an enzyme-labeled avidin is introduced additionally to react with biotin. A substrate solution is then introduced in a similar manner to the labeled enzyme, and the color development of the reacted substrate is detected and quantified. Yet alternatively when the label is a fluorescent dye, it is possible to determine the concentration of the marker contained in the specimen by measuring the fluorescence after a mixed solution containing the specimen and labeled antibody is fed to the reaction unit 4.

It is thus possible to perform immunoassay efficiently in a shorter period of time by using the microchip according to the present embodiment. Because the specimen and the labeled antibody are mixed before they are introduced to the micro-flow channel, it is also possible to integrate the steps of feeding specimen, washing solution and labeled antibody, which are conducted separately in conventional assays, into a single step, and simplify the procedure. For that reason, it is possible to shorten the period needed for reaction, reduce the possibility that foaming and impurities are mixed in the flow channel during reagent exchange between steps when the assay is performed in three separate steps, and also, to prevent fluctuation of the reaction.

### EXAMPLES

Hereinafter, the invention will be described with reference to an Examples and a Comparative Example.

### Example

A microchip 1 having a dam 5 in its micro-flow channel 3, as shown in FIG. 1, was prepared. An anti-CEA (CARCINOEMBRYONIC ANTIGEN) antibody was immobilized on polystyrene microbeads, and the microbeads surface not covered with the anti-CEA antibody was coated with a phosphate buffer solution containing 1% bovine serum albumin. The microbeads were introduced to the microchip to be filled into the micro-flow channel 3, forming a reaction unit 4.

A mixed solution containing CEA antigen and peroxidase-labeled antibody was fed previously into the microtube and allowed to react with the immobilized anti-CEA antibody. Then, a mixed solution of 3,3′,5,5′-Tetramethylbenzidine and H₂O₂ was introduced as substrates to perform the enzyme reaction. The substrate colored in the enzyme reaction was detected on a microchip for detection under TLM (excitation wavelength: 650 nm), and the color development was measured. Similar tests were repeated, while the antigen concentration was altered.

### Comparative Example

In a similar manner to the Example above, the experiments similar to that in the Example were conducted in the case where a solution of CEA antigen and a solution of peroxidase-labeled antibody are fed separately into the micro-flow channel 3 in the anti-CEA antibody-immobilized microchip.

Measurement results are summarized in Table 1. The TLM signals obtained in the Example, which were proportional to the antigen concentration, were higher in intensity than those in Comparative Example. The measurement period was also shortened from 27 minutes to 21 minutes.

TABLE 1
Unit (μV)

| CEA-antigen | | |
|---|---|---|
| concentration | Example | Comparative Example |
| 0 ng/mL | 839 | 198 |
| 0.5 ng/mL | 1,962 | 244 |
| 5 ng/mL | 3,222 | 291 |
| 50 ng/mL | 13,655 | 3,194 |
| Measurement period | 21 minutes | 27 minutes |

It is apparent that the present invention is not limited to the above embodiment, that may be modified and changed without departing from the scope and spirit of the invention.

1. An immunoassay method, which employs a sandwich method using a microchip having an antibody immobilized in the micro-flow channel thereof, comprising
feeding a fluid mixed with a labeled antibody and a specimen into the micro-flow channel.

2. The immunoassay method according to claim 1, wherein the label is an enzyme, a fluorescence dye, or biotin.
